SEQUENCE LISTING (8 sequences)

SEQ ID NO: 1 - DNA, 18 bp, synthetic construct
  source 1..18: mol_type = other DNA
gctgcctcccgtaggagt

SEQ ID NO: 2 - DNA, 18 bp, synthetic construct
  source 1..18: mol_type = other DNA
atccagctaccgtcaacc

SEQ ID NO: 3 - DNA, 18 bp, synthetic construct
  source 1..18: mol_type = other DNA
ccgtggctttctattccg

SEQ ID NO: 4 - DNA, 18 bp, synthetic construct
  source 1..18: mol_type = other DNA
ggcttccccatcggcatt

SEQ ID NO: 5 - DNA, 18 bp, synthetic construct
  source 1..18: mol_type = other DNA
tcccaagcattactcacc

SEQ ID NO: 6 - DNA, 18 bp, synthetic construct
  source 1..18: mol_type = other DNA
tagccgtccctttctggt

SEQ ID NO: 7 - DNA, 20 bp, synthetic construct
  source 1..20: mol_type = other DNA
agttatgcccgtatcgcctg

SEQ ID NO: 8 - DNA, 18 bp, synthetic construct
  source 1..18: mol_type = other DNA
gcctagatgccctttatg

(19) United States
(12) Patent Application Publication
(10) Pub. No.: US 2026/0078440 A1
(43) Pub. Date: Mar. 19, 2026

(54) IN VITRO CULTURE MODEL FOR BIOFILM STRUCTURAL ANALYSES

(71) Applicant: The Research Foundation for the State University of New York, Albany, NY (US)

(72) Inventor: Alex Valm, Albany, NY (US)

(21) Appl. No.: 19/194,417

(22) Filed: Apr. 30, 2025

Related U.S. Application Data
(60) Provisional application No. 63/640,307, filed on Apr. 30, 2024.

(51) Int. Cl. C12Q 1/6869; C12M 1/00; C12M 1/32; C12M 1/34; C12Q 1/6841

(52) U.S. Cl. CPC C12Q 1/6869; C12M 23/12; C12M 29/00; C12M 41/36; C12Q 1/6841

(57) ABSTRACT

A system and method to create an in vitro model of a biofilm structure. A fluid sample containing bacteria is collected and placed into a flow culture system that cultures the fluid sample in a media to produce cultured samples of the bacteria, which is then fed into a multispectral imager that multispectrally images the cultured samples. The multispectral imager performs linear unmixing of the multispectral images. The model allows testing specific hypotheses regarding systems-level community structure and investigation their effects on biofilm development and spatial structure. This system and model dental plaque and test the effects of various therapeutic interventions, such as antimicrobial agents, on the biofilm composition and structure.

### CROSS-REFERENCE TO RELATED APPLICATIONS

This application claims the benefit of U.S. Provisional Patent Application No. 63/640,307, filed Apr. 30, 2024, the entirety of which is hereby incorporated herein by this reference.

### STATEMENT REGARDING FEDERALLY SPONSORED RESEARCH

This invention was made with government support under grant number DE030927, awarded by the National Institutes of Health. The government has certain rights in the invention.

### SEQUENCE LISTING

The instant application contains a Sequence Listing which has been submitted via Patent Center and is hereby incorporated by reference in its entirety. Said .xml copy, created on Oct. 6, 2025, is named 010-23-11US01_SEQ, and is 8,075 bytes in size.

### BACKGROUND OF THE INVENTION

### 1. Field of the Invention

The present invention generally relates to in vitro biological models. More particularly, the present invention relates to a system and method for creating an in vitro model for growing in vitro biofilms and performing downstream multiplex labeling and multispectral imaging, particularly for modeling dental plaque.

### 2. Description of the Related Art

Microbial biofilms exist in a variety of niche environments of a human. Biofilms can also cause a range of infections and diseases because of their ability to evade host immune action and significant tolerance of antimicrobial agents. In attempting to devise effective antimicrobials and treatment strategies, the translation of their anti-biofilm activity from results obtained in in vitro studies in the lab to in vivo patient treatment success by currently available biofilm models.

Many biofilm studies performed using in vitro systems or animal models do not mimic or accurately represent the human host environment. The currently available in vitro models allow the collection of information in a manner that is high throughput, easy to use, flexible, controllable, and cost effective, but these studies can be limited or restricted in their translatability to biofilms present in vivo. Animal models present considerable ethical barriers, are complicated to run, require expertise, and cannot always accurately reflect human physiology and disease. As the general understanding of the biofilms present at various sites of infection increases, it is becoming clearer that the biofilms generated in many of the simpler and most commonly used in vitro models that fail to adequately consider the host environment.

For example, some basic biofilm models lack appropriate host factors, such as relevant substratum, flow/non-flow, host fluids, and the like, which have a profound impact on the biofilms formed in vivo, including their architecture/structure, gene expression, quorum sensing, virulence, and antimicrobial tolerance profiles. This has prompted the generation of several innovative and more advanced in vitro models that better recreate a variety of biofilm infected sites such as a chronic wound, acneic skin, fungal nail models, oropharynx, etc.

The current state of the art does not allow for continuous imaging of the biofilm throughout an experiment. Presently, live experiments must be ended in order to image the biofilm and extant systems use opaque medium making imaging difficult. It is thus to improve translatability in in vitro models that can better recapitulate the host environment that the present invention is primarily directed.

### SUMMARY OF THE INVENTION

Briefly described, the present invention provides a system and method that utilize a method for growing in vitro dental plaque biofilms and performing downstream multiplex labeling and multispectral imaging. Multispectral imaging allows for the simultaneous acquisition of multiple fluorescent signals, providing information on the spatial distribution of different bacterial taxa, in situ. This methodology enables the study of the dynamics of oral biofilm formation, the biophysical and biochemical interactions among different bacterial species, and the response of the biofilm to various treatments under controlled laboratory conditions.

The present invention includes the setup and growth of oral biofilms seeded with supragingival plaque of volunteers with an in vitro flow cell culture model. The in vitro dental plaque biofilms are harvested, labeled, multispectral imaged, and linear unmixed (LU). The multispectral image acquisition of model biofilms can be labeled with fluorescence in situ hybridization (FISH) probes the most abundant and prevalent genera in the human oral microbiome in combination with a FISH probe that labels most bacteria. The methodology can be adapted to a wide variety of biofilm models of clinical relevance provided taxon-specific probes are available.

In one embodiment, the system for modeling a biofilm with an in vitro cell culture includes at least one collector of a fluid sample, where the fluid sample contains bacteria, and there is a flow culture system that selectively receives the fluid sample from the collector and cultures the fluid sample in a media to produce one or more cultured samples of the bacteria in the fluid sample. There is a multispectral imager that selectively receives the cultured samples from the flow culture system and multispectrally images of the one or more cultured samples, with the multispectral imager further configured to perform linear unmixing of the multispectral images of the one or more cultured samples. The multispectral imager further outputs image data for the one or more cultured samples.

The system can include a DNA sequencer that selectively receives the fluid sample from the collector, with the DNA sequencer creating a DNA sequence for bacteria in the fluid sample. The system can also include a centrifuge that receives and centrifuges the fluid sample, with the centrifuged fluid sample is sent to the flow culture system. The multispectral imager can add fluorescence in situ hybridization probes prior to multispectrally imaging the one or more cultured samples.

The collector can be a microtube, and the fluid sample contains saliva, or can be a fluid tube, and the fluid sample contains dental plaque. The fluid tube can be a cryovial, and the system can further include a cryofreezer to preserve a fluid sample in the cryovial. Additionally, the flow collector can further include a fortified medium capable of culturing in vitro supragingival biofilms.

In an embodiment, the invention provides a method for structurally modeling a biofilm with an in vitro cell culture by the steps of collecting a fluid sample, where the fluid sample containing at least bacteria, then placing the fluid sample into a flow culture system that cultures the fluid sample in a media. The method continues with producing, at the flow culture system, one or more cultured samples of the bacteria in the fluid sample, and sending the one or more cultured samples to a multispectral imager. Then multispectrally imaging of the one or more cultured samples at the multispectral imager, and linear unmixing, at the multispectral imager, the multispectral images of the one or more cultured samples.

In one embodiment, the invention also provides a flow culture system, comprising: one or more inlets for selectively receiving a fluid sample, and an array of culture trays, each of which cultures the fluid sample in a media to produce one or more cultured samples of the bacteria in the fluid sample. There is an outlet to send the one or more cultures sample to another diagnostic device, such as a multispectral imager. The inlets can be configured to receive one or more microtubes containing fluid samples.

The present invention provides an advantage in the more accurate modeling of in vivo biofilms with an in vitro model. These in vitro models which include physical and computational components can better mimic the environments of a variety of clinically important, biofilm-associated infections of the skin, oropharynx, lungs, and infections. The present invention is further industrially applicable in that it can provide a model for effective treatment of bacterial biofilms, especially in medical treatments. Other objects features and advantages of the present invention will be apparent to one of skill in the art after review of the present application.

### BRIEF DESCRIPTION OF THE DRAWINGS

FIG. 1 is a pictorial diagram of the steps for one embodiment of the procedure to create an in vitro dental plaque model.

FIG. 2 is a pictorial diagram of one embodiment of a dental plaque on a chip flow cell setup.

FIG. 3 is an image of one embodiment of an in vitro dental plaque on a chip culture model.

FIG. 4 is a series of micrographs of oral biofilms cultured in dental plaque on a chip model.

### DETAILED DESCRIPTION OF THE INVENTION

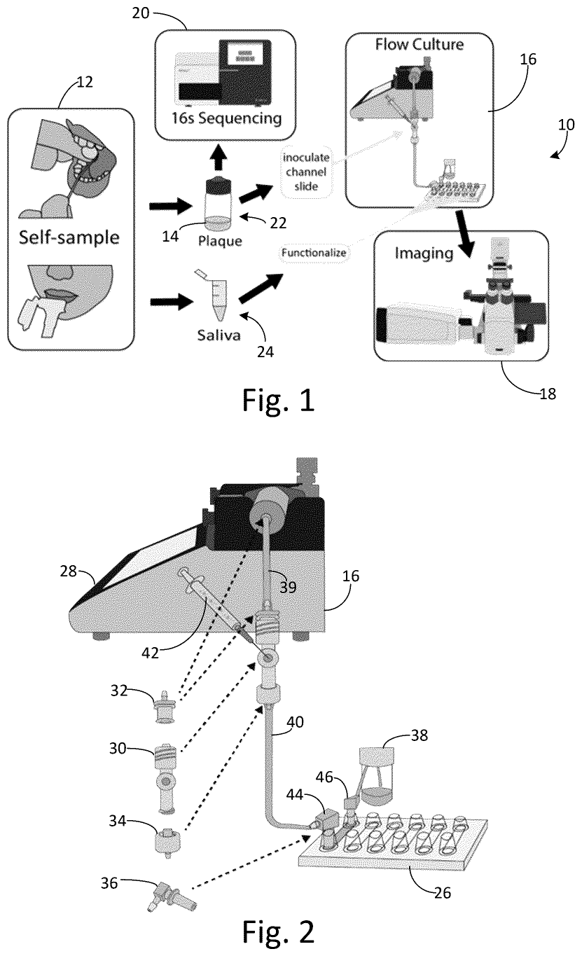

With reference to the drawings, in which like numbers represent like elements throughout, FIG. 1 is a pictorial diagram of the overview the system 10 for modeling a biofilm with an in vitro cell culture. There is at least one collector 12 of a fluid sample 14, where the fluid sample 14 contains bacteria, and there is a flow culture system 16 that selectively receives the fluid sample 14 from the collector 12 and cultures the fluid sample 14 in a media to produce one or more cultured samples of the bacteria in the fluid sample 14. The flow culture system 16 is more particularly illustrated in FIG. 2.

There is a multispectral imager 18 that selectively receives the cultured samples from the flow culture system 16 and multispectrally images of the one or more cultured samples, with the multispectral imager 18 further configured to perform linear unmixing of the multispectral images of the one or more cultured samples. The multispectral imager 18 further outputs image data for the one or more cultured samples. Such images are more specifically shown in FIG. 4.

The system 10 can include a DNA sequencer 20 that selectively receives the fluid sample from the collector 12, with the DNA sequencer 20 creating a DNA sequence for bacteria in the fluid sample. Other diagnostic devices can be used in addition to the DNA sequencer, such a microscopes, culturing media, and other chemical testing devices. The system 10 can also include a centrifuge (not shown) that receives and centrifuges the fluid sample, with the centrifuged fluid sample is sent to the flow culture system 12. The centrifuge can be commercial made, such as those sole by Benchmark®, Thermo Scientifc® and Eppendorf®. The multispectral imager 18 can add fluorescence in situ hybridization (FISH) probes prior to multispectrally imaging the one or more cultured samples, as is more fully described herein.

The collector 12 can be a microtube 24, and the fluid sample contains saliva, or can be a fluid tube 22, and the fluid sample contains dental plaque. The fluid tube 22 can be a cryovial, and the system 10 can further include a cryofreezer (not shown) to preserve a fluid sample 14 in the cryovial. The croyofreezer can be a commercially sold model such as those from Thermo Scientifc®, LABFENG®, and Cryomed®. Additionally, the flow collector can further include a fortified medium capable of culturing in vitro supragingival biofilms, as is more fully described herein.

As illustrated, in one embodiment, dental plaque is self-collected by a healthy volunteer, as shown at collector 12. In the laboratory, the sample is aliquoted and sent for DNA sequencing as DNA sequencer 20. Collected saliva (microtube 24) is used to functionalize a flow-cell microslide 26 (FIG. 2). A flow culture system 16 is constructed, and media is pumped via a syringe pump 28 (FIG. 2) over the functionalized microslide 26. The culture is inoculated with dental plaque (fluid jar 22) through an injection port 30 upstream of the slide and the system is incubated at 37° C. Biofilms are fixed and labeled for multispectral imaging.

FIG. 2 is a pictorial diagram of one embodiment of the flow culture system 16, which here is a dental plaque on a chip flow cell setup. FIG. 2 specifically illustrates syringe pump 28 for precise control of flow rate through the tube 40 into the microslide 26. There is a female Luer lock connector 34, injection port 30, male Luer lock connector 34, elbow Luer lock connector 36, all in the tube 40. Shown here is an Ibidi multichannel microslide 26. This, or another array of culture trays can be used in the present invention. Here, include is a fortified medium 38 capable of culturing in vitro supragingival biofilms added to the microslide 26.

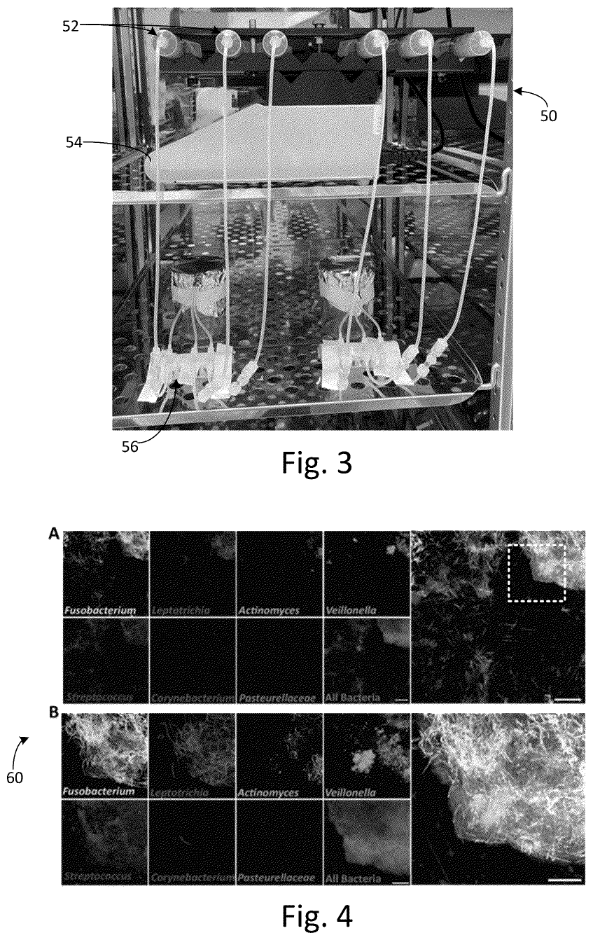

FIG. 3 is an image 50 of one embodiment of an in vitro dental plaque on a chip culture model. Six media containing syringes 52 are loaded into a multichannel syringe pump set 54 on a shelf above the multichannel flow slides 56. Each flow slide has three channels developing biofilms derived from the supragingival plaque of a single healthy donor. Flow slides 26 are placed on a slight incline rising towards waste receptacle to prevent bubble accumulation.

FIG. 4 are micrographs 60 of oral biofilms after Linear Unmixing (LU) by the multispectral imager. Micrograph A is oral biofilms cultured in dental plaque on a chip model, labeled for Fusobacterium, Leptotrichia, Actinomyces, Veillonella, Streptococcus, Corynebacterium, Pasteurellaceae, and all bacteria with EUB338 general bacterial probe. Linear unmixing allows abundance estimation of each fluorophore at every pixel. The scale bars, 20 μm. Micrograph B is a zoom field of view of the region highlighted with dashed lines in micrograph A. Scale bars, 10 μm.

In an embodiment, the model creation begins with dental plaque to preserve the microbial communities present in the dental plaque. Many dental plaque microbes are obligate anaerobes. Cryo-freezing medium should be pre-reduced and stored anaerobically before time of collection. Additionally, careful selection of fluorophores is vital for successful multispectral imaging. Here, 8 reference images in FIG. 4 are used of labeled Escherichia coli cells, one for each fluor that will be used in the final sample, for the purposes of extracting reference spectra that will be used for LU. In planning to conduct research using this in vitro model it is important to create a singly labeled sample for each fluorophore that will be present in the experimental sample.

In this embodiment, the equipment to start the protocol includes:
Equipment: 6-10 Channel Syringe Pump (e.g., Cole Parmer cat. no. 78-8200C); Gravity Incubator; Class II Biological Safety Cabinet
Materials: 1× μ-Slide VI 0.5 Glass Bottom (Ibidi, cat. no.: 80607); 2× Elbow Luer Connectors (Ibidi, cat. Nos.: 10802, 10826); 1× Luer Lock Connector, In-line Luer Injection Port, Luer Lock Coupler (Ibidi, cat. Nos.: 10826, 10820, 10823); 0.8 mm Silicone Tubing (Ibidi, cat. no.: 10841); 1×20 ml Luer-Lok Syringe sterile (e.g., BD, cat. no.: 302832) 3×1 ml Syringe with Needle (e.g., BD, cat. no.: BD 309597); 150 mL media bottle for waste receptacle.

In this embodiment, and with reference to FIG. 2, the ‘Dental Plaque on Chip Model Assembly and Growth Flow Cell Assembly’ begins by: 1. Attaching female Luer lock 32 connectors to each end of a 10-inch section of 0.8 mm silicone tubing 40 (FIG. 2). All aspects of flow cell assembly should be carried out in the sterile biological safety cabinet. 2. Attaching the In-line Luer Injection port 30 to the female Luer lock connector 32 on the compatible end. 3. Attaching the male Luer lock connector 34 to the opposite end of the In-line Luer Injection port 30. 4. Attach a 2-inch section of silicone tubing 40 to the male Luer lock connector 34. 5. Attach the male elbow Luer lock connector 36 to the opposite side of the 2-inch silicone tubing 40. 6. Attach female Luer lock coupler to end of male elbow Luer lock connector. 7. Attach the remaining male elbow Luer lock connector to the other section of the 10-inch silicone tubing. 8. Complete the line by attaching the male elbow Luer lock connector 36 to the open end of the female Luer lock coupler 44. 9. Place the end of the tubing assembly without the female Luer lock connector 44 into the waste receptacle. 10. Draw 20 ml of Gingival Margin (GM) media 38 into 20 ml BD Luer-Lok sterile syringe and attach to female Luer lock connector 46. Ensure air bubbles are removed from the media syringe 42 prior to assembly. 11. Prime flow setup with media. 12. Insert syringe(s) 42 (with tubing assembly complete) into the pump setup and set the flow rate to 5 μL/min, placing the syringe pump 28 setup above the flow cell and starting flow immediately (before adding Ibidi μ-Slide) helps prevent bubbles in the system. 13. In a biological safety cabinet, dilute donor saliva 1:1 with sterile DI water (total volume×mL) and spin down at 3220 RCF in a bench top swinging bucket centrifuge for 10 minutes to remove cells. 14. Pipette 50 ul of dilute donor saliva into Ibidi-Slide flow channel and incubate for 1 hour at room temperature under U.V. light with sash closed in a biological safety cabinet. This step functionalizes the coverslip surface in the μ-slide with salivary proteins and glycoproteins to form an adhesive surface for early colonizing plaque species. 15. In the 37° C. incubator, detach the male elbow Luer lock connectors 36 from the female Luer lock 44 coupler of running flow setup and securely attach to the Ibidi μ-Slide flow channel inlet followed by outlet (FIG. 3).

For the creation of an Inoculation and Microcosm Culture: 1. Rapidly thaw donor dental plaque (stored at −80° C.) in a 37° C. water bath and spin down at 8000 RCF for 5 minutes in a benchtop microcentrifuge to gently remove freezing medium, followed by 2× washes in mineral salt buffer (1.15 g/L Na₂HPO₄, 3.00 g/L NaCl, 0.20 g/L KCl, 0.20 g/L KH₂PO₄, 0.10 g/L MgSO₄·7H₂O). Rehydrate washed dental plaque in 50 μl of mineral salt buffer for each biofilm to be grown (e.g., for six biofilms, rehydrate in 300 μl of mineral salt buffer). Do not spin cells live cells at higher speed than 8000 RCF, this could damage the cells and differentially reduce viability. 2. Using a small binder clip clamp the tubing 39 upstream of the injection port 32. Draw 50 μl of dental plaque solution into a sterile syringe and inject into the injection port 32, then remove the clamp to restart flow. Clamping the tubing line 39 upstream of the injection port during plaque inoculation prevents dental plaque contamination of the GM media upstream of the inoculation port 30. 3. Repeat steps 1 & 2 above at 3 hours and 6 hours for a total of three inoculations.

Dental plaque samples that are used to seed the in vitro model system can obtained from volunteers through dental flossing. Volunteers are instructed to abstain from oral hygiene (i.e., tooth brushing, flossing, and using mouthwash) for 48 hours, after which informed consent is confirmed, and samples are collected by laboratory personnel. Volunteers are asked to floss between 12 teeth with 12 separate pieces of dental floss and subsequently expectorate 10 mL of saliva into a sterile collection tube. Each piece of floss containing dental plaque is split into two aliquots and immediately stored at −80° C. for future use. Saliva collected from volunteers is centrifuged and stored at −20° C. in a cryofreezer for later flow cell hybridization.

In a further embodiment, the equipment includes: Benchtop microcentrifuge; Benchtop swinging bucket centrifuge Anaerobic chamber; Class II biological safety cabinet; a cryofreezer. The materials: Ice; bucket with ice; Sterile gloves; 50 ml conical tube (VWR Cat. No. 82050); 1.5 mL microcentrifuge tube (VWR Cat. No. 10025); Sterile PBS solution (Fisher Cat. No. 10010023); Dental floss (Benco Dental Cat. No. 4799-917); Bottled potable water; Pre-reduced Liquid Dental Transport (LDT) media (Anaerobe Systems Cat. No. AS-916); Anaerobic glycerol (Fisher Cat. No. 35-635-2100ML); 2 μm filters (e.g., Fisher Cat. No. 720-1320); 50 ml syringe (e.g., VRW Cat. No. BD309653); 15 mL conical tube; Screw cap silicone gasket cryovials (e.g., Fisher Scientific Cat. No. 10-269-88A); Dry ice; >99% ethanol.

For preparation of 50% anaerobic glycerol-DTM: 1. Transport 500 mL glycerol bottle, 2 μm filters, 50 ml syringe and ˜25×1 ml bottles of LDT to the anaerobic chamber. Inside the chamber, loosen the cap on the glycerol bottle. 2. Using a pipettor, transfer 20 mL of LDT to a 50 ml conical tube. Carefully pour 20 mL of glycerol to the tube. Vortex the tube vigorously to suspend the glycerol. 3. Place a 2 μm filter on the end of a 50 ml syringe. 4. Pour the 50% glycerol mixture into the syringe. 5. Use the plunger to force the 50% glycerol solution through the filter. Collect in a sterile filter cap 50 ml tube. Invert the tube 3×, then vortex for 60 seconds to mix. Keep this mixture in the anaerobic chamber for long term use. This medium should be prepared up to 6 months before collecting oxygen-sensitive dental plaque and saliva samples and kept in the anaerobic chamber until use.

For sample collection and storage: 1. Instruct the person to floss between 12 pairs of teeth, (3 pairs of teeth in all 4 quadrants) and deposit each piece of floss in a sterile 1.5 mL microcentrifuge tube, then close the lid. 4. Instruct the patient to expectorate 10 mL of unstimulated saliva in a sterile 50 ml conical tube with the 10 mL mark highlighted. The saliva tube is kept on ice during the collection procedure, which may take up to 1 hour or more depending. 5. Immediately upon completion of collection, process plaque and saliva samples for long-term storage.

For the cryopreservation of dental plaque samples: 1. Transport the 12 dental floss tubes into the anaerobic chamber. 2. To the first tube, pipet 1 mL LDT. Pipet up and down vigorously 20 times to dislodge all the dental plaque from the floss. Close the lid on the tube and vortex mix for 10 seconds. 3. Open the tube, remove the floss and discard in a dedicated autoclave waste bag. 4. Transfer the 1 mL of plaque solution to the next dental floss tube and repeat step 3 until you have resuspended 6 tubes. 5. After 6 tubes, start over with a fresh 1 mL of LDT and collect the plaque from the remaining 6 tubes. 6. Combine both plaque suspensions into one 15 ml conical tube. 7. Pipette 2 mL 50% glycerol/50% LDT to the plaque suspension to create a 25% glycerol solution. Vortex mix for 5 seconds. 8. Aliquot 200 μL of this glycerol plaque suspension into each of 20 screwcap, silicone gasket cryovials. 9. Screw the cap on tight on the cryovials and remove from chamber. It is important to screw the cap and form a tight seal to prevent both oxygen and ethanol from entering the tube in subsequent steps. 10. Prepare a dry ice-ethanol bath. Prepare bath in the chemical fume hood and wear appropriate personal protective gear including lab coat, cryo-gloves, and face shield to prevent supercooled ethanol from contacting skin. 11. Wait 10 minutes for the dry ice-ethanol bath to reach a low boil. Drop each of the 20 tubes in a test tube rack in the dry ice ethanol bath. Let them sit there for 5-10 minutes to snap freeze. Make sure the tubes are not fully submerged in the ethanol bath. The cap should be above the ethanol to prevent contamination of the sample with ethanol. 12. After 10 minutes, remove the cryovials carefully using forceps and place in a freezer box. Place the box in a −80 cryofreezer for storage up to 1 year.

For cryopreservation of saliva cells and preservation of saliva: 1. In a bench-top swinging bucket centrifuge, spin the 50 mL saliva tube at 12,000 RPM for 20 minutes at 4° C. 2. Transport the 50 mL tube containing human saliva to the biological safety cabinet. 3. Open the tube and decant the saliva into a sterile 50 mL centrifuge tube. Use caution, the separated saliva may stick tightly to the cell pellet. If the cell pellet accidentally gets disturbed, recombine pellet with supernatent and spin it again for 20 min in centrifuge. 4. Store cell-free saliva at −20° C. for up to 1 year. 5. Transport the saliva cell pellet to the anaerobic chamber. 6. Resuspend the pellet in 2 mL LDT. Pipet up and down vigorously 20 times. Vortex mix for 10 seconds. 7. Add 2 mL 50% glycerol-50% LDT. Pipet up and down 10 times and vortex mix for 5 seconds. 8. Aliquot 200 μL of the cell pellet glycerol slurry into each of 20 screwcap cryovials. Screw the cap with silicone gasket on tightly. 9. Drop each of the 20 tubes in the dry ice ethanol bath prepared as described previously. Allow the pellets to freeze solid in the bath. 10. After 20-30 minutes, remove the cryovials carefully using forceps and place in −80° C. cryofreezer for long-term storage.

For a second support protocol for Gingival Margin (GM) Medium Preparation (such as medium 38, the GM medium is adapted from Shi medium, a fortified medium capable of culturing in vitro supragingival biofilms closely resembling the diversity of the initial inoculum. GM medium is modified to better recapitulate the nutritional milieu experienced by microbes at the gingival margin (transitional zone). Sheep's blood is replaced with heat-inactivated human serum. To induce inter-cellular metabolic cooperation, components of Shi media that presumably are derived not from the host but rather from other members of the microbiota, i.e., N-acetyl muramic acid and vitamin K are not included and the final media is diluted to 50% strength in DI water. After medium preparation is complete, the media should be filter sterilized to remove large mucin particles which non-specifically bind and sequester FISH probes and interfere with downstream quantitative image analysis.

For the materials: sterile 10 ml syringe (e.g., BD 301029); sterile 0.2 μm PES syringe filter (e.g., Corning 431229); 150 mL sterile disposable 0.2 μm PES filter unit (e.g., Thermoscientific cat. no. 5650020). Hemin Solution: Hemin (Sigma, cat. no.: 51280); K₂HPO₄ (Fisher, cat. no.: P288-50); MiliQ H₂O.

For the base media: Protease peptone (Fisher, cat. no.: BP1420-500); Trypticase peptone (BD Bacto, cat. no.: 211705); Yeast extract (BD Bacto, cat. no.: 212750); Porcine gastric mucin (Sigma, cat. no.: M1778); KCl (Fisher, cat. no.: P2175) urea (Fisher, cat. no.: U15-500); L-arginine (Fisher, cat. no.: BP370-100); L-lysine (Fisher, cat. no.: BP386-100); Glycine (Fisher, cat. no.: BP381-500); Sucrose (Fisher, Cat. No. A15583-36); Heat Inactivated Human Serum (Sigma, cat. no.: H3667). Note: Serum should be stored at −20° C. and only be added to the media at the time of the experiment.

For the GM media preparation protocol: 1. Prepare hemin solution in advance by combining 10 mg Hemin, 1.74 g K₂HPO₄, and 100 ml MiliQ H2O, in flask and heat to boiling. Store in 10 ml aliquots at −20° C. 2. Prepare the base media stock by combining 10 g protease peptone, 3 g trypticase peptone, 5 g yeast extract, 2.5 g porcine gastric mucin, 2.5 g KCl, 0.06 g urea, 0.87 g L-arginine, 0.182 g L-lysine, 0.075 g glycine, and 1.0 g sucrose in a 2 L flask followed by the addition of 970 ml of DI H2O and add 10 ml of Hemin solution. 3. Dilute the media 1:1 in DI H2O and autoclave at 121° C. for 45 minutes. Allow to cool to below 60° C. 4. Filter sterilize media to remove large mucin particles in a 150 mL filter unit and store at 4° C. until needed. 5. At time of experiment, retrieve 120 mL of GM media and warm to 37° C. 6. Thaw and filter sterilize 6 mL of human serum in a 10 ml syringe with 0.2 μm filter attached and add to GM media just prior to use.

For the protocol for microcosm labeling and multispectral image acquisition: Equipment: Hybridization oven capable of adjustable temperature in range of 46-48° C.; Laser scanning confocal microscope equipped with spectral detector(s) (e.g., Zeiss LSM 710, 880, 980; Nikon A1; Leica SP5, SP8; and others). Materials: Fixation: 4% Paraformaldehyde (PFA) (Electron Microscopy Sciences cat. no. 157-4); Phosphate-buffered saline (PBS, Fisher cat. no. 10010023); Ethyl alcohol; 0.75″ paper binder clip (e.g., Staples 10667-CC). FISH buffer: 5 M NaCl; 1 M Tris pH 7.5; 1% sodium dodecyl sulfate (SDS); HiDi formamide (Thermofisher Cat #4440753); Milli-Q ultrapure water. For FISH Probes (See Table 1 for probe sequences and fluorophore characteristics): AF488-ACT476; AF514-VEI488; AF555-FUS714; RRX-PAS111; AF594-STR405; AF647-COR633; AF660-LEP568; PacBlue-EUB338. For the Wash Buffer 1:5 M NaCl; 1 M Tris pH 7.5; 1% sodium dodecyl sulfate (SDS); HiDi formamide (Thermofisher Cat. No. 4440753); Milli-Q ultrapure water. For Wash Buffer 2:5 M NaCl; 1 M Tris pH 7.5; 1% sodium dodecyl sulfate (SDS); Milli-Q ultrapure water; SlowFade Gold Antifade Mountant (Invitrogen cat. no. S36940)

For the Microcosm Fixation and FISH Labeling Protocol: 1. Clamp silicone tubing 40 with 0.75″ binder clip directly upstream and downstream of Ibidi μ-Slide flow channel 26, followed by the careful removal of Luer lock elbow connector 36. Attach each end of Luer lock elbow connector 36 to Luer lock coupler so that remainder of setup breakdown can be done later. Clamping tubing 40 prior to removal of mircoslide 26 from flow setup helps to preserve fragile biofilm from being disturbed by mechanical stress and media flow during removal from setup. 2. Gently pipette 60 μL of 4% PFA solution into each biofilm containing flow channel, angling the pipette tip towards Ibidi μ-Slide reservoir wall. Cover with included Ibidi μ-Slide lid and allow to fix for 1.5 hours at room temperature. Angling pipette tip towards reservoir wall serves to preserve fragile biofilm. After removal of elbow Luer lock connectors, there is approximately 60 μL of media volume left over in the channel/reservoir. The addition of 60 μL 4% PFA yields a final concentration of 2% PFA. NOTE: The total combined volume of channel and reservoir is 160 μL. 3. Remove approximately 60 μL of PFA solution by pipetting from channel outlet. Subsequently, wash flow channel twice by using 120 μL of PBS solution followed by twice with a 1:1 ethanol to PBS solution. Seal with male Luer lock plugs and store at 4° C. for at least 24 hours prior to labeling. 4. Remove Luer lock plugs and remove approximately 80 μL of ethanol PBS solution from channel outlet. Subsequently pipette 120 μL of wash buffer 1 into inlet followed by removal from outlet (repeat twice). 5. Remove 80 μL of wash 1 solution from channel outlet and pipette 120 μL of FISH Buffer. Remove an additional 60 μL from channel outlet followed by the addition of 60 μL of FISH Buffer. Seal with Luer lock plug and place in hybrid oven at 46° C. overnight. Replace FISH buffer with wash buffer 1 as previously described and incubate at 48° C. for 15 minutes. Replace wash buffer 1 with wash buffer 2 and incubate at 48° C. for an additional 10 minutes. 6. Dehydrate by pipetting 2×120 μL each of 50% EtOH, 70% EtOH, and 100% EtOH. Remove all remaining EtOH in flow channel and allow flow channel to dry completely. 7. Mount in SlowFade Gold Antifade Mountant (pipet approximately 100 μL into channel).

For the multispectral image acquisition and linear unmixing: 1. Use microscope ocular to find a section of biofilm containing all label types. This may require searching via live scan modes for fluorescent labels that are not visible to the eye under epifluorescence. This step is required in order to select microscope settings to optimally excite all fluorophores present in the sample without saturating pixels. 2. Begin setting the image acquisition parameters in scan mode. Optimize acquisition settings starting with the shortest wavelength laser (488 nm). This excitation wavelength will maximally excite AF488, AF514. Set the laser power such that the dynamic range in the image is maximized but no pixels are saturated in any spectral channel. Leaving on the 488 nm laser, repeat the process with the 561 nm laser to excite AF555, AF594, RRX. Leaving both 488 nm and 561 nm laser on, repeat again with the 633 nm laser to excite AF647 and AF660. 3. Determine the number of channels to record. When using a 32-element detector, the maximum number of channels is 32. Not all channels may be necessary for the specific experiment. For example, the shortest excitation wavelength in this protocol is 488 nm, so it is not necessary to collect wavelengths below 488 nm. 4. Acquire spectral images of singly labeled samples by labelling E. coli samples with PacBlue, AF488, AF514, AF555, RRX, AF594, AF647 and AF660. The singly labeled samples will be acquired with identical settings as the multi-labeled image. 5. Extract reference spectra from the singly labelled E. coli cell images for each of the seven fluorophores used in the experiment. Reference spectra will be used as inputs for a linear unmixing (LU) algorithm. 6. Apply LU to the multi-labeled biofilm sample. 7. After unmixing determine the quality of the image visually and determine if settings need to be adjusted. If settings are adjusted, then repeat steps 5 and 6.

The human oral microbiome is a diverse community of fungi, viruses and over 700 known species of bacteria, a subset of which live as members of the dental plaque biofilm on tooth surfaces. Compositional changes in dental plaque microbiome communities in the transition from health to disease, termed dysbiosis have been well-documented; however, changes in the spatial structure of these communities during disease progression have not been well studied. Mapping the systems level structure of dental plaque biofilms is critical for understanding the spatio-temporal and functional changes that occur in oral microbial communities during the transition from health to periodontal disease and caries.

In vitro oral microcosm communities, seeded with mixed communities of human dental plaque have proven to be an essential tool for studying the taxonomic composition of dental plaque communities and the changes in community composition in response to specific perturbations. Compositional analyses of these models have shown them to support the growth of many dozens of organisms in co-culture. However, these models are usually grown in media that may not be representative of human saliva or gingivocrevicular fluid and may be grown on optically opaque surfaces, e.g., hydroxyapatite or other materials, e.g., plastic that are incompatible with high resolution, multispectral fluorescence imaging.

Several parameters affect the ability to map the spatial structure of oral microcosms cultured via the methods described in this protocol. When assembling and priming the biofilm flow system, it is critical to minimize the introduction and formation of bubbles in the lines. Bubble formation may be unavoidable during, e.g., cellular respiration, therefore it is critical to orient the flow cell at an angle to sequester bubbles in one area of the flow cell. For FISH probe choice, the user may rely on known abundance and prevalence information in human dental plaque; however, the high inter-individual variability that has been reported in this community may justify DNA sequencing of replicate biofilms to identify organisms of interest for targeting with FISH probes.

For robust and accurate linear unmixing using reference standards as described here, it is important that the biofilm images be acquired on the microscope using the same image acquisition settings as the reference images. In practice, this means that the acquisition settings should first be set using the biofilm sample, on a region of the heterogeneous community where all fluorescent labels are present. The dynamic range of the multispectral image should be maximized for each excitation laser used. Once these settings are optimized, the same settings should be used for acquiring reference images. In practice, reference images may be higher intensity than the biofilm image. Saturation in pixels must be avoided as it is incompatible with linear unmixing. If reference images are much brighter than the biofilm images and saturation is present in the recorded image, the excitation energy must be decreased in such a way that all spectral channels are decreased proportionately. This may be achieved through: 1. decreasing the pixel dwell time or 2. decreasing the laser power proportionately for each laser, but the user should be aware that laser power output is linear over a finite range of power settings on the instrument, and is expected to be non-linear towards the minimal and maximal power settings.

To interpret the results, biofilms harvested after 24-48 hours of growth are expected to have taxonomic diversity similar to the initial inoculum, with some taxa overrepresented and others under-represented or completely absent (FIG. 4). The culture conditions described here are designed to recapitulate the in vivo formation of dental plaque on teeth; however, some components are missing from this model, e.g., the host immune system which is known to influence oral biofilm composition. Fluorescence intensity in the recorded image may be low for some taxa if those organisms were not metabolically active at the time of biofilm fixation, but in general, the rich media supplied here and the availability of unoccupied niches on the slide surface for biofilm cell attachment should promote the growth of diverse and metabolically active microbes. After fixation, biofilms are stable and can be stored for 6 months or more at 4° C. in 50% ethyl alcohol, though care should be taken to ensure that biofilms do not dry during storage. Inter-individual heterogeneity in dental oral microbiome composition, especially at the strain level will result in heterogeneous in vitro biofilms. Dental plaque samples from multiple donors may be pooled before inoculation to reduce variability from biological replicates.

For time considerations: Prior to in vitro flow culture setup: Filter sterilization of mucin containing medium is slow and therefore this step should be done before running the in vitro flow cell experiment. In this protocol we use a Nalgene Rapid Flow bottle top filters with 50 mm diameter 2 μm PES membrane filter. Each filter can sterilize approximately 30 ml of dilute GM medium [˜5 mins/30 ml]. On day of in vitro flow culture setup [˜8 hours]: Sufficient time [˜1 hour] should be allocated for flow system setup prior to hybridization of saliva to μ-slide channels [1 hour]; this allows for media to reach 37° C. during saliva hybridization step. Inoculation of donor plaque into each flow channel occurs at 0 hours, 3 hours and 6 hours [6 hours]. All culturing steps can be completed in a day.

Acquisition of spectral z-stacks in thick biofilm sections requires additional time compared to nonspectral imaging and time must also be allocated for LU of spectral images. Approximately 30 minutes are required for the acquisition of each z-stack assuming similar settings to that used for the image in FIG. 3 (Scan Mode: LineSequential, Pixel Time: 1.24 μs, Scan Direction: Unidirectional, Averaging: 2, Lamda mode: 32 channels, image size: 1584 by 1584 pixels, pixel scaling: 0.09 μm×0.09 μm×0.320 μm, Z-stack: 127 Slices).

TABLE 1
Salient characteristics of fluorescence in situ hybridization probes for 7 genera of bacteria
abundant in dental plaque and for all bacteria as used in the protocol here.

| | | Peak | Peak | | | | | |
|---|---|---|---|---|---|---|---|---|
| | | Exci- | Emi- | | | | SEQ | |
| Fluoro- | Abbrev- | tation | ssion | Probe | | | ID | Ref- |
| phore | iation | λ (nm) | λ (nm) | Name | Target | Sequence | NOs. | erence |
| Pacific | PacBlue | 410 | 455 | EUB338 | All bacteria | GCTGCCTCCCGTAGGA | 1 | Amann |
| Blue | | | | | | GT | | 1990 |
| Alexafluor | AF488 | 495 | 519 | ACT476 | Actinomyces | ATCCAGCTACCGTCAA | 2 | Gmur |
| 488 | | | | | | CC | | 2004 |
| Alexafluor | AF514 | 517 | 542 | VEI488 | Veillonella | CCGTGGCTTTCTATTC | 3 | Chalmers |
| 514 | | | | | | CG | | 2008 |
| Alexafluor | AF555 | 555 | 565 | FUS714 | Fusobacterium | GGCTTCCCCATCGGCA | 4 | Valm |
| 555 | | | | | | TT | | 2011 |
| Rhodamine | RRX | 560 | 580 | PAS111 | Pasteurellaceae | TCCCAAGCATTACTCA | 5 | Valm |
| Red-X | | | | | | CC | | 2011 |
| Alexafluor | AF594 | 590 | 617 | STR405 | Streptococcus | TAGCCGTCCCTTTCTG | 6 | Paster |
| 594 | | | | | | GT | | 1998 |
| Alexafluor | AF647 | 650 | 665 | COR633 | Corynebacterium | AGTTATGCCCGTATCG | 7 | Valm |
| 647 | | | | | | CCTG | | 2011 |
| Alexafluor | AF660 | 663 | 690 | LEP568 | Leptotrichia | GCCTAGATGCCCTTTA | 8 | Valm |
| 660 | | | | | | TG | | 2011 |

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A system for modeling a biofilm with an in vitro cell culture, comprising:
at least one collector of a fluid sample, the fluid sample containing bacteria;
a flow culture system that selectively receives the fluid sample from the collector and cultures the fluid sample in a media to produce one or more cultured samples of the bacteria in the fluid sample; and
a multispectral imager that selectively receives the one or more cultured samples from the flow culture system and multispectrally images of the one or more cultured samples, the multispectral imager further configured to perform linear unmixing of the multispectral images of the one or more cultured samples.

2. The system of claim 1, wherein the multispectral imager further outputs image data for the one or more cultured samples.

3. The system of claim 1, a DNA sequencer that selectively receives the fluid sample from the collector, the DNA sequencer creating a DNA sequence for bacteria in the fluid sample.

4. The system of claim 1, further including a centrifuge that receives and centrifuges the fluid sample, and the centrifuged fluid sample is sent to the flow culture system.

5. The system of claim 1, wherein the collector is a microtube, and the fluid sample contains saliva.

6. The system of claim 1, wherein the collector is a fluid tube, and the fluid sample contains dental plaque.

7. The system of claim 1, wherein the multispectral imager adds fluorescence in situ hybridization probes prior to multispectrally imaging the one or more cultured samples.

8. The system of claim 6, wherein the fluid tube is a cryovial, and further including a cryofreezer to preserve a fluid sample in the cryovial.

9. The system of claim 1, wherein the flow collector further including a fortified medium capable of culturing in vitro supragingival biofilms.

10. A method for structurally modeling a biofilm with an in vitro cell culture, comprising:
collecting a fluid sample, the fluid sample containing at least bacteria;
placing the fluid sample into a flow culture system that cultures the fluid sample in a media;
producing, at the flow culture system, one or more cultured samples of the bacteria in the fluid sample;
sending the one or more cultured samples to a multispectral imager;
multispectrally imaging of the one or more cultured samples at the multispectral imager; and
linear unmixing, at the multispectral imager, the multispectral images of the one or more cultured samples.

11. The method of claim 10, further outputting, from the multispectral imager, image data for the one or more cultured samples.

12. The method of claim 10, further:
receiving, at a DNA sequencer, the fluid sample from the collected fluid sample; and
creating a DNA sequence, at the DNA sequencer, for bacteria in the fluid sample.

13. The method of claim 10, further:
receiving, at a centrifuge, the fluid sample;
centrifuging the fluid sample; and
sending the centrifuged fluid to the flow culture system.

14. The method of claim 10, wherein collecting a fluid sample is collecting a fluid sample containing saliva.

15. The method of claim 10, wherein collecting a fluid sample is collecting a fluid sample containing dental plaque.

16. The method of claim 10, further adding, at the multispectral imager, fluorescence in situ hybridization probes prior to multispectrally imaging the one or more cultured samples.

17. The method of claim 10, further including cryofreezing the fluid sample.

18. The method of claim 10, further culturing the fluid sample by culturing the fluid sample in a fortified medium capable of culturing in vitro supragingival biofilms.

19. A flow culture system, comprising:
one or more inlets for selectively receiving a fluid sample containing bacteria;
an array of culture trays, each of which cultures the fluid sample in a media to produce one or more cultured samples of the bacteria in the fluid sample; and
an outlet to send the one or more cultures sample to another diagnostic device.

20. The system of claim 19, wherein the inlets are configured to receive one or more microtubes containing fluid samples.
